(19) United States
(12) Patent Application Publication
(10) Pub. No.: US 2005/0118234 A1
(43) Pub. Date: Jun. 2, 2005

(54) Methods and kits related to administration of a fructooligosaccharide

(75) Inventors: Gregory Dean Sunvold, Lewisburg, OH (US); Thomas William-Maxwell Boileau, Springfield, OH (US); Robert Jason Vickers, Dayton, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The IAMS Company

(21) Appl. No.: 10/724,839

(22) Filed: Dec. 1, 2003

(51) Int. Cl. A61K 35/78; A61K 31/715

(52) U.S. Cl. 424/442; 424/764; 514/54

(57) ABSTRACT

A first embodiment disclosed herein is a method of enhancing total tract digestibility of one or more dietary components in a companion animal, the method comprising administering to the companion animal a companion animal composition comprising fructooligosaccharide. Kits comprising the companion animal composition and information that use of the companion animal composition by a companion animal is useful for enhancing total tract digestibility of one or more dietary components in the companion animal, are also disclosed. In a related, but separate, embodiment, a method selected from enhancing calcium absorption, improving bone health, improving strength, improving physical activity performance, and combinations thereof, the method comprising administering to a companion animal a companion animal composition comprising fructooligosaccharide, is disclosed. Kits comprising the companion animal composition and information that use of the companion animal composition by a companion animal is useful for a purpose selected from the group consisting of enhancing calcium absorption, improving bone health, improving strength, improving physical activity performance, and combinations thereof, are also disclosed.

### FIELD OF THE INVENTION

The present invention is directed to methods of using companion animal compositions comprising fructooligosaccharide, as well as kits comprising such compositions.

### BACKGROUND OF THE INVENTION

The anatomy, physiology, and microbial ecology of the mammalian gastrointestinal tract differs among humans, dogs, cats, and other species. For example, when normalized to body length or weight, total length of the canine and feline gastrointestinal tract is shorter than that of omnivorous mammals such as humans, and because of transit time differences and dietary patterns, the bacterial flora of the intestine also varies. Since the bacterial flora of the intestine is primary responsible for the breakdown of fermentable fibers such as fructooligosaccharide, it is expected that different species will uniquely utilize fermentable fibers.

Few studies have directly compared the ability of dogs, cats and humans to utilize dietary fibers. One study has compared the ability of the bacterial flora of dog, cat and human large intestine to metabolize several dietary fibers. Butyrate production from the tested fermentable fibers, which is characteristic of fiber metabolism, was about 50% greater for the human samples compared to either cat or dog and lactate production was about 73% lower for cat and human compared to dog samples. See Sunvold et al., “In vitro fermentation of cellulose, beet pulp, citrus pulp, and citrus pectin using fecal inoculum from cats, dogs, horses, and pigs and ruminant fluid from cattle,” Journal of Animal Science, Vol. 73, pp. 3639-3648 (1995). Thus, it is clear from this study that: 1) companion animals differ from humans in their ability to metabolize and utilize dietary fibers including fructooligosaccharide; and 2) that biological effects of dietary fibers will vary between dogs, cats and humans based on these differences, and that studies utilizing humans will not necessarily be predictive of findings in companion animals such as dogs and cats.

It has been surprisingly discovered herein that companion animals utilize fermentable fiber in a unique manner, for example relative to nutrient digestibility and calcium absorption, thereby leading to novel therapies for treating gastrointestinal health, improving bone health, and other treatments. Even further, based on the research of the present inventors, previous work related to companion animals such as dogs may have been incomplete in terms of the beneficial effects of fermentable fiber relative to nutrient digestibility and related action. For example, using small numbers of dogs, previous work has indicated that total tract nutrient digestibility is not affected through administration of fermentable fiber in the dog. See e.g., Flickinger et al., “Nutrient digestibilities, microbial populations, and protein catabolites as affected by fructan supplementation of dog diets,” J. Anim. Sci., Vol. 81, pp. 2008-2018 (2003). However, surprisingly, based on the further study by the inventors, action such as nutrient digestibility has now been shown to be affected through use of fermentable fiber in the companion animal, including the dog. This and other benefits of the present invention are described herein.

### SUMMARY OF THE INVENTION

The present invention is directed to a variety of embodiments, wherein a first embodiment is a method of enhancing total tract digestibility of one or more dietary components in a companion animal, the method comprising administering to the companion animal a companion animal composition comprising fructooligosaccharide. Kits comprising the companion animal composition and information that use of the companion animal composition by a companion animal is useful for enhancing total tract digestibility of one or more dietary components in the companion animal, are also provided.

In a related, but separate, embodiment, the invention is directed to a method selected from enhancing calcium absorption, improving bone health, improving strength, improving physical activity performance, and combinations thereof, the method comprising administering to a companion animal a companion animal composition comprising fructooligosaccharide. Kits comprising the companion animal composition and information that use of the companion animal composition by a companion animal is useful for a purpose selected from the group consisting of enhancing calcium absorption, improving bone health, improving strength, improving physical activity performance, and combinations thereof, are also provided.

### DETAILED DESCRIPTION OF THE INVENTION

Various documents including, for example, publications and patents, are recited throughout this disclosure. All such documents are hereby incorporated by reference.

All percentages and ratios are calculated by weight unless otherwise indicated. All percentages and ratios are calculated based on the total composition unless otherwise indicated.

Referenced herein are trade names for components including various ingredients utilized in the present invention. The inventors herein do not intend to be limited by materials under a certain trade name. Equivalent materials (e.g., those obtained from a different source under a different name or reference number) to those referenced by trade name may be substituted and utilized in the descriptions herein.

In the description of the invention various embodiments and/or individual components are disclosed. As will be apparent to the ordinarily skilled practitioner, all combinations of such embodiments and components are possible and can result in preferred executions of the present invention.

The compositions herein may comprise, consist essentially of, or consist of any of the components as described herein.

While various embodiments and individual components of the present invention have been illustrated and described, various other changes and modifications can be made without departing from the spirit and scope of the invention. As will be also be apparent, all combinations of the embodiments and components taught in the foregoing disclosure are possible and can result in preferred executions of the invention.

### Methods and Kits of the Present Invention

The presently inventive methods and kits utilize companion animal compositions comprising a fructooligosaccharide. Fructooliogosaccharides are naturally-occurring compounds which can be found in a variety of fruits or vegetables including banana, barley, garlic, honey, onion, rye, brown sugar, tomato, asparagus, artichoke, wheat, yacon, or chicory. Fructooligosaccharide may for example be provided as chicory, as inulin, or as short chain oligofructose. Particularly useful herein are fructooligosaccharide comprising at least one of 1-kestose (abbreviated as GF₂), nystose (GF₃), and 1F-beta-fructofuranosylnystose (GF₄). While fructooligosaccharides can be extracted from plants such as those mentioned herein, they can also be formed artificially by adding one, two, or three fructose units to a sucrose molecule by a B-(2-1)-glycosidic linkage of the fructose unit(s) to the fructose unit of sucrose. As an example, fructooligosaccharides are commercially available under the tradename NUTRAFLORA from Golden Technologies Company, Incorporated (which is a short chain oligofructose comprising 1-kestose, nystose, and 1F-beta-fructofuranosylnystose. As another example, a mixture of short chain fructooligosaccharide and inulin can be PREBIO1 or a mixture of commercially available RAFTILOSE and RAFTILINE.

The fructooligosaccharide may be a short chain oligofructose, which will be well-known to those of ordinary skill in the art. Particularly useful herein are short chain oligofructose comprising 1-kestose (abbreviated as GF₂), nystose (GF₃), and 1F-beta-fructofuranosylnystose (GF₄). In a preferred embodiment, the short chain oligofructose comprises from about 25% to about 45% 1-kestose, from about 25% to about 45% nystose, and from about 1% to about 20% 1F-beta-fructofuranosylnystose, by weight of the short chain oligofructose, alternatively from about 30% to about 40% 1-kestose, from about 50% to about 60% nystose, and from about 5% to about 15% 1F-beta-fructofuranosylnystose, by weight of the short chain oligofructose. As an example, short chain oligofructose is commercially available under the tradename NUTRAFLORA from Golden Technologies Company, Incorporated (which is a short chain oligofructose comprising about 35% 1-kestose, 55% nystose, and 10% 1F-beta-fructofuranosylnystose, all by weight of the short chain oligofructose).

In an embodiment herein, the fructooligosaccharide may display certain organic matter disappearance percentages. In this optional embodiment, the fructooligosaccharide may have an organic matter disappearance (OMD) of from about 15% to about 60% when fermented by fecal bacteria in vitro over a 24 hour period. That is, from about 15% to about 50% of the total organic matter originally present is fermented and converted by the fecal bacteria. The organic matter disappearance of the fructooligosaccharide is alternatively from about 20% to about 50%, alternatively from about-30% to about 40%.

Thus, in vitro OMD percentage may be calculated as follows:
(1−((OM residue−OM blank)/original OM))×100
where OM residue is the organic matter recovered after 24 hours of fermentation, OM blank is the organic matter recovered in corresponding blank tubes (i.e., tubes containing medium and diluted feces, but no substrate), and original OM is that organic matter placed into the tube prior to fermentation. Additional details of the procedure are found in Sunvold et al., J. Anim. Sci., Vol. 73, pp. 1099-1109 (1995).

The companion animal compositions may comprise various levels of fructooligosaccharide, as described herein. For example, the compositions may comprise from about 0.01% to about 10%, alternatively from about 0.01% to about 5%, alternatively from about 0.01% to about 2%, alternatively from about 0.01% to about 2%, alternatively from about 0.01% to about 0.15% of fructooligosaccharide, all by weight of the companion animal composition.

As stated, the fructooligosaccharide utilized herein may be chicory or inulin. Alternatively, in an optional embodiment herein, the compositions are substantially free of inulin and/or chicory (also commonly referenced as chicory root). As used herein, “substantially free of,” with reference to the material, means that the composition comprises less than about 0.1% of the referenced material, more preferably less than about 0.05% of the referenced material, even more preferably less than about 0.01% of the referenced material, even more preferably less than about 0.005% of the referenced material, all by weight of the composition.

Optionally, the composition herein may be a food composition such as a dry composition (for example, kibble), semi-moist composition, wet composition, or any mixture thereof. Alternatively or additionally, the composition is a supplement, such as a gravy, drinking water, yogurt, powder, suspension, chews, treats (e.g., biscuits) or any other delivery form.

Moreover, in a preferred embodiment the composition is nutritionally balanced. As used herein, the term “nutritionally balanced,” with reference to the companion animal composition, means that the composition has known required nutrients to sustain life in proper amounts and proportion based on recommendations of recognized authorities in the field of companion animal nutrition.

The compositions herein may optionally comprise one or more further components. Other components are beneficial for inclusion in the compositions used herein, but are optional for purposes of the invention. For example, as stated, food compositions are preferably nutritionally balanced. In one embodiment, the food compositions may comprise, on a dry matter basis, from about 20% to about 50% crude protein, alternatively from about 20% to about 40% crude protein, by weight of the food composition, or alternatively from about 20% to about 35% crude protein. The crude protein material may comprise vegetable proteins such as soybean, cottonseed, and peanut, or animal proteins such as casein, albumin, and meat protein. Non-limiting examples of meat protein useful herein include a protein source selected from the group consisting of beef, pork, lamb, poultry, fish, vegetable, and mixtures thereof.

Furthermore, the compositions may comprise, on a dry matter basis, from about 5% to about 40% fat, alternatively from about 10% to about 35% fat, by weight of the food composition.

The compositions of the present invention may further comprise a source of carbohydrate. Grains or cereals such as rice, corn, milo, sorghum, barley, alfalfa, wheat, and the like are illustrative sources.

The compositions may also contain other materials such as dried whey and other dairy by products.

The compositions may further comprise a fiber source additional to the short chain oligofructose. A variety of soluble or insoluble fibers may be utilized, which will be well-known to those of ordinary skill in the art. In one embodiment, at least a portion of the fiber source is selected from beet pulp (from sugar beet), gum arabic, gum talha, psyllium, rice bran, carob bean gum, citrus pulp, pectin, fructooligosaccharide, mannanoligofructose, soy fiber, arabinogalactan, galactooligosaccharide, arabinoxylan, and mixtures thereof.

In one embodiment, the additional fiber source comprises a fermentable fiber. Fermentable fibers are not digested by mammals but may be metabolized by intestinal bacterial species, such as Bifidobacterium. However, not all intestinal bacteria can metabolize fermentable fiber. In particular, bacteria such as Salmonella, E. coli and Clostridia are unable to process such fiber to any meaningful degree. This preferential digestibility, which is applicable for fermentable fiber as a class, can be used to improve the overall bacterial flora in the small intestine of the companion animal. Because fermentable fibers will only feed “good” bacteria such as Lactobacillus and Bifidobacterium, the amounts of harmful bacteria such as Salmonella, E. coli and Clostridia may decrease due to a reduction in food resources. Therefore, by providing a preferred food source for beneficial bacterial species, a diet supplemented with fermentable fiber can increase “good” intestinal bacteria while reducing the amount of “bad” bacteria.

The compositions may optionally comprise a total dietary fiber level of from about 0.001% to about 30%, alternatively from about 0.01% to about 20%, or alternatively from about 1% to about 16% total dietary fiber, by weight of the composition.

The compositions may also comprise one or more nutrients. Consistent with the benefits associated with enhanced calcium absorption herein, one of ordinary skill may optionally lower the levels of calcium relative to that which is present in typical, or premium, companion animal compositions. For example, optionally, the compositions comprise less than about 1% calcium, by weight of the composition, alternatively from about 0.01% to about 0.95% calcium, alternatively from about 0.1% to about 0.95% calcium, alternatively from about 0.5% to about 0.95% calcium, all by weight of the composition.

### Methods of the Present Invention

The methods of the present invention comprise administering (i.e., through ingestion) a composition of the present invention to a companion animal to provide one or more of the health benefits described herein.

In one embodiment, the invention relates to methods of enhancing total tract digestibility of one or more dietary components in a companion animal. Total tract digestibility is well-known in the art, and methods of determining total tract digestibility, including total tract ash digestibility, total tract fiber digestibility, total tract fat digestibility, and total tract dry matter digestibility are described herein below. The dietary component may be any dietary component which the ordinarily skilled artisan desires for enhancement, examples of which may be a nutrient or other component of food. In one embodiment, the method relates to enhancing total tract digestibility of a plurality of dietary components.

In another embodiment, the invention relates to methods selected from enhancing calcium absorption, improving bone health, improving strength, improving physical activity performance, and combinations thereof. While each of these benefits, and methods of measurement will be well-known to those of ordinary skill in the art, various methods of analysis directed to several of these benefits are described herein below.

Generally, enhancement of calcium absorption is well-known in the art. Moreover, bone health benefits include, but are not limited to, preventing, inhibiting, ceasing, and/or reversing bone loss and/or building bone mass, and/or preventing, inhibiting, ceasing, and/or reversing osteoporosis. Moreover bone health benefits include preventing, treating and/or reducing the severity of any one of a number of bone disorders specific to companion animals (hip dysplasia, osteoarthritis, mobility issues, and the like). Thus, improved bone health may provide, for example, healthy bones, stronger bones, and/or increased bone mass. See e.g., Ohta et al., “A Combination of Dietary Fructooligosaccharides and Isoflavone Conjugates Increases Femoral Bone Mineral Density and Equol Production in Ovariectomized Mice,” The Journal of Nutrition, July 2002, pp. 2048-2053; Mineo et al., “Various Indigestible Saccharides Enhance Net Calcium Transport from the Epithelium of the Small and Large Intestine of Rats In Vitro,” The Journal of Nutrition, December 2001, pp. 3243-3246; Takahara et al., “Fructooligosaccharide Consumption Enhances Femoral Bone Volume and Mineral Concentrations in Rats,” The Journal of Nutrition, July 2000, pp. 1792-1795; Morohashi et al., “True Calcium Absorption in the Intestine is Enhanced by Fructooligosaccharide Feeding in Rats,” The Journal of Nutrition, October 1998, pp. 1815-1818.

The inventors herein have further discovered the importance of the fructooligosaccharide component described herein relative to strength and physical activity performance of a companion animal. Without intending to be limited by theory, it is believed that the enhanced calcium absorption herein demonstrated with respect to the companion animal upon ingestion of the composition comprising fructooligosaccharide is a precursor to enhancements in both strength and physical activity performance. Indeed, this is supported by known activity of calcium relative to muscle contraction and related mechanisms. Strength and physical performance are described in a variety of diverse fields of art, and is readily measurable by various techniques and ready experimentation. See e.g., U.S. Patent Application Publication 2003/0194478 and U.S. Pat. No. 6,117,872.

As used herein, the term “administering” with respect to the companion animal means that the animal ingests or a human is directed to feed, or does feed, the animal one or more compositions herein. Wherein the human is directed to feed the composition, such direction may be that which instructs and/or informs the human that use of the composition may and/or will provide the referenced benefit, for example, enhancing total tract digestibility. For example, such direction may be oral direction (e.g., through oral instruction from, for example, a veterinarian or other health professional), radio or television media (i.e., advertisement), or written direction (e.g., through written direction from, for example, a veterinarian or other health professional (e.g., scripts), sales professional or organization (e.g., through, for example, marketing brochures, pamphlets, or other instructive paraphernalia), written media (e.g., internet, electronic mail, or other computer-related media)), and/or packaging associated with the composition (e.g., a label present on a container holding the composition). As used herein, “written” means through words, pictures, symbols, and/or other visible descriptors. Such information need not utilize the actual words used herein, for example, “total”, “tract”, or “absorption”, but rather use of words, pictures, symbols, and the like conveying the same or similar meaning are contemplated within the scope of this invention.

The compositions described herein may be used as a supplement to ordinary dietetic requirements or may serve as the primary food for the companion animal (and, as such, the supplements or foods may be nutritionally balanced). Administration may be on as as-needed or as-desired basis, for example, once-monthly, once-weekly, or daily (including multiple times daily). When utilized as a supplement to ordinary dietetic requirements, the composition may be administered directly to the companion animal or otherwise contacted with or admixed with companion animal food. When utilized as a companion animal food, administration will be well-known to those of ordinary skill. The amount of composition utilized may be dependent on a variety of factors, including the quality of gastrointestinal health of the animal, preference of the animal as determined by the guardian of the animal or other person administering the composition, the quality of the companion animal food, and size or breed or the companion animal.

### Methods of Analysis

Various methods may be utilized to determine the action fructooligosaccharide will have in relation to a given method of use. Non-limiting examples follow:

Methods of Determining Tract Digestibility

Methods of analysis for determining tract digestibility of one or more dietary components will be well-known to those of ordinary skill in the art. As a non-limiting example, the following methods may be utilized in such regard:

Animals and Diets. In a crossover design, healthy adult dogs (n=56) are randomly assigned to 1 of 2 groups: Group 1, 21 days of feeding a diet containing 0% fructooligosacharride (0% FOS) followed by 28 days of feeding a diet containing 0.15% fructooligosaccharide (0.15% FOS): and Group 2, 21 days of feeding a diet containing 0% FOS followed by 28 days of feeding a diet containing 0.25% fructooligosaccharide (0.25% FOS). Dogs are provided meals and water at 2 time points during the day and the uneaten food and water is recorded.

Sampling Procedures. Procedures for this experiment are conducted under a research protocol which is approved for the protection of the health and well-being of animals. Each period of the study includes an adaptation phase (16 day for 0% FOS and 23 day for 0.15% and 0.25% FOS) followed by a five-day total excreta collection phase. During the adaptation phase food amounts are adjusted to maintain weight balance. During the five-day collection period, dogs are housed in stainless steel metabolism cages and all cages are equipped with a wire-mesh floor and urine drip pan to allow separation of feces and urine. During the entire experiment, dogs are in a temperature controlled room (20° C.), with a 15-hour dark/9-hour light cycle. Dietary metabolizable energy (ME) values and calculated ME requirements (based on weight changes) are used to establish daily food allowances for the collection phase. All dogs are offered one-half of their daily allowance each day at 0645 and 1200, and any feed refusals are collected, weighed, and discarded. Each dog is offered 1600 mL of water each day. Dogs are weighed weekly during the experiment.

Urine samples are collected in vessels containing 20 mL of 6 N HCL to prevent microbial growth and ammonia volatilization. The daily urine output is measured, saved, and composited for subsequent laboratory analyses. Feces are collected, weighed, and scored for consistency daily, with 100 g saved and composited. Composited fecal samples are lyophilized for 3-d, ground (2-mm screen; Wiley mill), and subsequently analyzed to determine nutrient digestibility.

Chemical Analyses. Feed and fecal samples are analyzed for dry matter (DM), ash, and nitrogen (N) (Association of Official Analytical Chemist Official Method, 1995). Fat content of feed (2 g) and feces (1 g) is determined using acid hydrolysis (Association of Official Analytical Chemist Official Method, 1995) for 45 minutes at 75.5° C. in 25% HCL (10 mL) followed by ether extraction (120 mL). Total dietary fiber (TDF) concentrations in the feed and fecal samples are determined by the AOAC method. Gross energy determinations of feed, fecal, and urine samples are conducted using a bomb calorimeter (Parr Instrument, Moline, Ill.). Protein digestibility (%) is calculated on a crude protein (CP) basis. Total tract digestibility is calculated using the following formulas:
Total Tract Digestibility={(dietary intake−total fecal nutrient output)/dietary intake}×100%

Calcium is measured in diet, feces and urine using inductively coupled plasma mass spectrometry.

Calcium retention is determined using the following formula:
Calcium Absorption=(dietary calcium intake−(fecal calcium+urine calcium))

Urine samples are prepared for energy determination by drying a 3 to 4 g sample at 105° C. in a forced-air oven. The dried samples are combined with a benzoic acid tablet prior to combustion. The GE value of each sample is corrected for the addition of benzoic acid (AOAC, 1995).

### Methods of Making

The presently described companion animal compositions are made according to methods which will be well known by the ordinarily skilled artisan. To illustrate, the compositions of the present invention may be prepared by mixing all components singularly or in suitable combinations together, and in water where appropriate, agitating mechanically until all of the ingredients have been solubilized, dispersed, or otherwise mixed, as applicable. Wherein certain processes such as extrusion (to form kibbles, for example) are utilized, such processes will be well-known in the art.

### EXAMPLES

The following are non-limiting examples of the present compositions which are prepared utilizing conventional methods. The following examples are provided to illustrate the invention and are not intended to limit the scope thereof in any manner.

### Example 1

Two kibble compositions having the following components at the approximate indicated amounts are prepared using methods which are standard in the art and are fed to dogs, each resulting in improved gastrointestinal health and improved fecal odor:

| | Example 1A | Example 1B |
|---|---|---|
| | (Component | (Component |
| | Amount indicated | Amount indicated |
| Component | as Wt %) | as Wt %) |
| Short Chain Oligofructose | 0.19 | 0.15 |
| (NUTRAFLORA, commercially | | |
| available from Golden | | |
| Technologies Company, | | |
| Incorporated | | |
| Poultry, Poultry By-product | 44 | 47 |
| Meal, and Fish Meal | | |
| Animal Fat | 8 | 6 |
| Beet Pulp | 2 | 3 |
| Salts | 2.5 | 2 |
| Vitamins and Minerals* | 1 | 1 |
| Minors | 3.5 | 4 |
| Grains | Remainder | Remainder |
| (corn, sorghum, barley, rice) | | |

*Vitamins and Minerals include: Vitamin E, beta-carotene and Vitamin A, Zinc Oxide, Ascorbic Acid, Manganese Sulfate, Copper Sulfate, Manganous Oxide, Calcium Pantothenate, Biotin, Vitamin B₁₂, Vitamin B₁, Niacin, Vitamin B₂, Vitamin B₆, Vitamin D₃, Folic Acid.

### Example 2

Total tract digestibility following administration of the kibble foods of Example 1 are each tested in the dog. As the samples tested are foods, the foods each contain a plurality of dietary components. The test is conducted in accordance with the descriptions provided herein above with respect to total tract digestibility, and specifically ash digestibility, fiber digestibility, fat digestibility, and dry matter digestibility. Trending increases in each of ash digestibility, fiber digestibility and dry matter digestibility are shown with the kibble food of Example 1A, with significant increases in fat digestibility with this kibble food. Significant increases in each of ash digestibility, fiber digestibility, fat digestibility, and dry matter digestibility are shown with the kibble food of Example 1B. These results are unexpected relative to the art.

### Example 3

Calcium absorption following administration of the kibble foods of Example 1 is tested in the dog. As the samples tested are foods, the foods each contain a plurality of dietary components. The test is conducted in accordance with the descriptions provided herein above with respect to calcium absorption. Significant increases in calcium absorption are shown with the kibble foods of both Example 1A and Example 1B. Moreover, following administration of the kibble foods of Example 1, the dogs exhibit increased strength and physical performance as observed according to a variety of known models. These results are unexpected relative to the art.

### Example 4

A beef-flavor gravy composition is prepared by combining the following components in a conventional manner:

| Component | Wt % |
|---|---|
| Fructooligosaccharide | 5.3 |
| Chicken Fat | 3.0 |
| Spray-Dried Beef Particles and Broth | 3.0 |
| Beet Pulp | 0.4 |
| Xanthan Gum | 0.5 |
| Flax Seed | 0.2 |
| Vegetables | 0.2 |
| Vitamins | 0.06 |
| Minerals | 0.04 |
| Phosphoric Acid | 0.95 |
| Beef Flavor | 0.1 |
| Water | Remainder |

The gravy composition is admixed with standard dog kibble diet daily prior to feeding to a growing canine in order to improve calcium absorption and bone health following administration of the kibble and gravy. Amounts of the gravy composition are determined as desired by the guardian of the dog.

### Example 5

A chicken-flavor gravy composition is prepared by combining the following components in a conventional manner:

| Component | Wt % |
|---|---|
| Short Chain Oligofructose (NUTRAFLORA, | 5.3 |
| commercially available from GTC Nutrition, | |
| Golden, CO., U.S.A.) | |
| Chicken Fat | 3.0 |
| Spray-Dried Chicken Particles and Broth | 3.0 |
| Beet Pulp | 0.4 |
| Xanthan Gum | 0.5 |
| Flax Seed | 0.15 |
| Vegetables | 0.2 |
| Vitamins | 0.06 |
| Minerals | 0.04 |
| Phosphoric Acid | 0.95 |
| Chicken Flavor | 0.53 |
| Water | Remainder |

The gravy composition is admixed with standard dog kibble diet daily prior to feeding to a dog in order to improve total tract digestibility of the kibble and gravy dietary components. Amounts of the gravy composition are determined as desired by the guardian of the dog.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method of enhancing total tract digestibility of one or more dietary components in a companion animal, the method comprising administering to the companion animal a companion animal composition comprising fructooligosaccharide.

2. The method according to claim 1 which is a method of enhancing total tract digestibility of a plurality of dietary components.

3. The method according to claim 2 wherein the companion animal composition comprises from about 0.01% to about 10% fructooligosaccharide, by weight of the composition.

4. The method according to claim 3 which is a method of enhancing total tract digestibility selected from the group consisting of total tract ash digestibility, total tract fiber digestibility, total tract fat digestibility, total tract dry matter digestibility, and combinations thereof.

5. The method according to claim 4 wherein the companion animal composition comprises from about 0.01% to about 5% fructooligosaccharide, by weight of the composition.

6. The method according to claim 5 wherein the fructooligosaccharide comprises inulin.

7. The method according to claim 5 wherein the fructooligosaccharide comprises chicory.

8. The method according to claim 5 wherein the fructooligosaccharide is a short chain oligofructose comprising from about 30% to about 40% 1-kestose, from about 50% to about 60% nystose, and from about 5% to about 15% 1F-beta-fructofuranosylnystose, by weight of the short chain oligofructose.

9. The method according to claim 8 wherein the companion animal composition comprises from about 0.01% to about 2% fructooligosaccharide, by weight of the composition.

10. The method according to claim 9 wherein the companion animal composition is a nutritionally balanced food.

11. The method according to claim 10 wherein the companion animal composition further comprises a protein source selected from the group consisting of beef, pork, lamb, poultry, fish, vegetable, and mixtures thereof.

12. The method according to claim 11 wherein the companion animal composition comprises from about 0.01% to about 0.2% fructooligosaccharide, by weight of the composition.

13. The method according to claim 12 wherein the companion animal composition is administered at least once daily.

14. The method according to claim 13 wherein the companion animal is a dog.

15. The method according to claim 13 wherein the companion animal is a cat.

16. The method according to claim 13 wherein the companion animal composition further comprises a fiber source additional to the fructooligosaccharide.

17. The method according to claim 16 wherein at least a portion of the fiber source is selected from the group consisting of fructooligosaccharide additional to the short chain oligofructose, beet pulp, gum arabic, gum talha, psyllium, rice bran, carob bean gum, citrus pulp, pectin, mannanoligofructose, soy fiber, arabinogalactan, galactooligosaccharide, arabinoxylan, and mixtures thereof.

18. A kit comprising:
(a) a companion animal composition comprising fructooligosaccharide; and
(b) information that use of the companion animal composition by a companion animal is useful for enhancing total tract digestibility of one or more dietary components in a companion animal.

19. The kit according to claim 18 comprising information that use of the companion animal composition by a companion animal is useful for enhancing total tract digestibility of a plurality of dietary components in the companion animal.

20. The kit according to claim 19 wherein the companion animal composition comprises from about 0.01% to about 5% fructooligosaccharide, by weight of the composition.

21. The kit according to claim 20 wherein the fructooligosaccharide comprises inulin.

22. The kit according to claim 20 wherein the fructooligosaccharide comprises chicory.

23. The kit according to claim 20 wherein the fructooligosaccharide is a short chain oligofructose comprising from about 30% to about 40% 1-kestose, from about 50% to about 60% nystose, and from about 5% to about 15% 1F-beta-fructofuranosylnystose, by weight of the short chain oligofructose.

24. The kit according to claim 23 wherein the companion animal composition comprises from about 0.01% to about 2% fructooligosaccharide, by weight of the composition.

25. The kit according to claim 24 wherein the companion animal composition is a nutritionally balanced food.

26. The kit according to claim 25 wherein the companion animal composition further comprises a protein source selected from the group consisting of beef, pork, lamb, poultry, fish, vegetable, and mixtures thereof.

27. The kit according to claim 26 wherein the companion animal composition comprises from about 0.01% to about 0.2% fructooligosaccharide, by weight of the composition.

28. The kit according to claim 27 wherein the companion animal is a dog.

29. The kit according to claim 27 wherein the companion animal is a cat.

30. The kit according to claim 27 wherein the companion animal composition further comprises a fiber source additional to the fructooligosaccharide.

31. The kit according to claim 30 wherein at least a portion of the fiber source is selected from the group consisting of fructooligosaccharide additional to the short chain oligofructose, beet pulp, gum arabic, gum talha, psyllium, rice bran, carob bean gum, citrus pulp, pectin, mannanoligofructose, soy fiber, arabinogalactan, galactooligosaccharide, arabinoxylan, and mixtures thereof.

32. A method selected from the group consisting of enhancing calcium absorption, improving bone health, improving strength, improving physical activity performance, and combinations thereof, the method comprising administering to a companion animal a companion animal composition comprising fructooligosaccharide.

33. The method according to claim 32 wherein the companion animal is selected from the group consisting of dogs, cats, and combinations thereof.

34. The method according to claim 33 which is selected from the group consisting of enhancing calcium absorption, improving bone health, and combinations thereof.

35. The method according to claim 34 wherein the companion animal composition comprises from about 0.01% to about 5% fructooligosaccharide, by weight of the composition.

36. The method according to claim 35 wherein the fructooligosaccharide comprises inulin.

37. The method according to claim 35 wherein the fructooligosaccharide comprises chicory.

38. The method according to claim 35 wherein the fructooligosaccharide is a short chain oligofructose comprising from about 30% to about 40% 1-kestose, from about 50% to about 60% nystose, and from about 5% to about 15% 1F-beta-fructofuranosylnystose, by weight of the short chain oligofructose.

39. The method according to claim 38 wherein the companion animal composition comprises from about 0.01% to about 2% fructooligosaccharide, by weight of the composition.

40. The method according to claim 39 wherein the companion animal composition is a nutritionally balanced food.

41. The method according to claim 40 wherein the companion animal composition comprises from about 0.01% to about 0.2% fructooligosaccharide, by weight of the composition.

42. The method according to claim 41 wherein the companion animal composition is administered at least once daily.

43. The method according to claim 42 wherein the companion animal composition comprises from about 0.01% to about 5% fructooligosaccharide, by weight of the composition.

44. The method according to claim 43 wherein the fructooligosaccharide comprises inulin.

45. The method according to claim 43 wherein the fructooligosaccharide comprises chicory.

46. The method according to claim 43 wherein the fructooligosaccharide is a short chain oligofructose comprising from about 30% to about 40% 1-kestose, from about 50% to about 60% nystose, and from about 5% to about 15% 1F-beta-fructofuranosylnystose, by weight of the short chain oligofructose.

47. The method according to claim 33 which is a method selected from the group consisting of improving strength, improving physical performance, and combinations thereof.

48. The method according to claim 47 wherein the companion animal composition comprises from about 0.01% to about 5% fructooligosaccharide, by weight of the composition.

49. The method according to claim 48 wherein the fructooligosaccharide comprises inulin.

50. The method according to claim 48 wherein the fructooligosaccharide comprises chicory.

51. The method according to claim 48 wherein the fructooligosaccharide is a short chain oligofructose comprising from about 30% to about 40% 1-kestose, from about 50% to about 60% nystose, and from about 5% to about 15% 1F-beta-fructofuranosylnystose, by weight of the short chain oligofructose.

52. The method according to claim 51 wherein the companion animal composition comprises from about 0.01% to about 2% fructooligosaccharide, by weight of the composition.

53. The method according to claim 52 wherein the companion animal composition is a nutritionally balanced food.

54. The method according to claim 53 wherein the companion animal composition comprises from about 0.01% to about 0.2% fructooligosaccharide, by weight of the composition.

55. The method according to claim 54 wherein the companion animal composition is administered at least once daily.

56. A kit comprising:
(a) a companion animal composition comprising fructooligosaccharide; and
(b) information that use of the companion animal composition by a companion animal is useful for a purpose selected from the group consisting of enhancing calcium absorption, improving bone health, improving strength, improving physical activity performance, and combinations thereof.

57. The kit according to claim 56 wherein the companion animal is selected from the group consisting of dogs, cats, and combinations thereof.

58. The kit according to claim 57 wherein the companion animal composition comprises from about 0.01% to about 5% fructooligosaccharide, by weight of the composition.

59. The kit according to claim 58 wherein the fructooligosaccharide comprises inulin.

60. The kit according to claim 58 wherein the fructooligosaccharide comprises chicory.

61. The kit according to claim 60 wherein the fructooligosaccharide is a short chain oligofructose comprising from about 30% to about 40% 1-kestose, from about 50% to about 60% nystose, and from about 5% to about 15% 1F-beta-fructofuranosylnystose, by weight of the short chain oligofructose.

62. The kit according to claim 61 wherein the companion animal composition comprises from about 0.01% to about 2% fructooligosaccharide, by weight of the composition.

63. The kit according to claim 62 wherein the companion animal composition comprises from about 0.01% to about 0.2% fructooligosaccharide, by weight of the composition.
